(19) United States
(12) Patent Application Publication
(10) Pub. No.: US 2022/0400994 A1
(43) Pub. Date: Dec. 22, 2022

(54) VIDEO-BASED PHYSIOLOGICAL SENSING INTEGRATED INTO VIRTUAL CONFERENCES

(71) Applicant: Microsoft Technology Licensing, LLC, Redmond, WA (US)

(72) Inventors: Javier Hernandez RIVERA, Sommerville, MA (US); Daniel J. MCDUFF, Cambridge, MA (US); Jin A. SUH, Seattle, WA (US); Kael R. ROWAN, Box Elder, SD (US); Mary P. CZERWINSKI, Kirkland, WA (US)

(73) Assignee: Microsoft Technology Licensing, LLC, Redmond, WA (US)

(21) Appl. No.: 17/349,000

(22) Filed: Jun. 16, 2021

(51) Int. Cl. A61B 5/16; A61B 5/0205; A63F 13/21; A61B 5/00

(52) U.S. Cl. CPC A61B 5/165; A61B 5/0205; A63F 13/21; A61B 5/0022

(57) ABSTRACT

Systems and methods for providing a user characteristic to a service provider for a virtual conference with a user are provided. In particular, a computing device may collect raw media data associated with the user during the virtual conference between the user and the service provider. During the virtual conference, the computing device may perform a first processing of the raw media data to extract intermediate user data, wherein the intermediate user data comprises one or more of a physiological signal and a behavioral signal associated the user. The computing device may further transform the raw media data into transformed media data and transmit the transformed media data with the intermediate user data to a server for second processing of the intermediate user data.

### BACKGROUND

Virtual conferencing systems (e.g., teleconferencing systems and videoconferencing systems) allow multiple parties to communicate with each other from remote locations. As technologies around virtual conferencing systems has evolved in recent years, a number of appointments held via virtual conference has increased due to flexibility and time savings offered by such virtual visits. For example, a user may participate in a virtual visit (e.g., medical appointments or personal fitness training sessions) via videoconference with a third-party (e.g., a healthcare provider and/or a fitness trainer). However, in most scenarios, the third parties lack access to objective measurements of the user's condition because of the inability to measure physiological and/or behavioral characteristics of the user during a virtual conference.

In some cases, user data indicative of the physiological and/or behavioral characteristics of the user may be collected by a user device and transmitted to a server device to be analyzed. However, there is a potential risk for a data loss during the transmission of the user data, which may result in lower accuracy of the measurements of the user's condition.

It is with respect to these and other general considerations that the aspects disclosed herein have been made. Also, although relatively specific problems may be discussed, it should be understood that the examples should not be limited to solving the specific problems identified in the background or elsewhere in this disclosure.

### SUMMARY

In accordance with at least one example of the present disclosure, a method for providing a user characteristic to a service provider for a virtual conference with a user is provided. The method may include collecting, by a user computing device, raw media data associated with the user during the virtual conference between the user and the service provider, performing, by the user computing device during the virtual conference, a first processing of the raw media data to extract intermediate user data, wherein the intermediate user data comprises one or more of a physiological signal and a behavioral signal associated the user, transforming, by the computing device, the raw media data into transformed media data, and transmitting, by the computing device, the transformed media data with the intermediate user data to a server for a second processing of the intermediate user data.

In accordance with at least one example of the present disclosure, a method for providing a user characteristic to a service provider for a virtual conference with a user is provided. The method may include collecting, by a user computing device, media data associated with the user, performing, by the user computing device, a first processing of the media data to extract intermediate user data, transmitting, by the user computing device, the media data with the intermediate user data to a server, performing, by the server, a second processing of the media data and the intermediate user data to generate the user characteristic, and providing, by the server, the user characteristic to the service provider for use in the virtual conference.

In accordance with at least one example of the present disclosure, a system for providing a user characteristic to a service provider for a virtual conference with a user is provided. The system may include a computing device and a server. The computing device may be configured to collect media data associated with the user, perform a first processing of the media data to extract intermediate user data, the intermediate user data comprising one or more of a physiological signal and a behavioral signal associated with the user, transmit the media data with the intermediate user data to a server. The server may be configured to perform a second processing of the media data and the intermediate user data to generate the user characteristic and provide the user characteristic to the service provider for use in the virtual conference.

Any of the one or more above aspects in combination with any other of the one or more aspects. Any of the one or more aspects as described herein.

This Summary is provided to introduce a selection of concepts in a simplified form, which is further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the following description and, in part, will be apparent from the description, or may be learned by practice of the disclosure.

### BRIEF DESCRIPTION OF THE DRAWINGS

Non-limiting and non-exhaustive examples are described with reference to the following Figures.

FIG. 1 depicts details directed to a virtual conferencing system in accordance with examples of the present disclosure;

FIGS. 2 and 3 depict a method directed to providing user characteristics to a service provider for a virtual conference in accordance with examples of the present disclosure;

FIG. 4 depicts block diagram illustrating physical components (e.g., hardware) of a computing device with which aspects of the disclosure may be practiced;

FIG. 5A illustrates a first example of a computing device with which aspects of the disclosure may be practiced;

FIG. 5B illustrates a second example of a computing device with which aspects of the disclosure may be practiced; and

FIG. 6 illustrates at least one aspect of an architecture of a system for processing data in accordance with examples of the present disclosure.

### DETAILED DESCRIPTION

In the following detailed description, references are made to the accompanying drawings that form a part hereof, and in which are shown by way of illustrations specific aspects or examples. These aspects may be combined, other aspects may be utilized, and structural changes may be made without departing from the present disclosure. Aspects may be practiced as methods, systems or devices. Accordingly, aspects may take the form of a hardware implementation, an entirely software implementation, or an implementation combining software and hardware aspects. The following detailed description is therefore not to be taken in a limiting sense, and the scope of the present disclosure is defined by the appended claims and their equivalents.

In accordance with examples of the present disclosure, the invention uses computer vision and audio processing to leverage sensors, such as cameras and microphones, to collect media data that is ultimately processed to extract physiological and/or behavioral characteristics of a user. These user characteristics may include features such as peripheral blood flow, heart rate, heart rate variability, respiration, blood oxygenation, blood pressure, facial actions, body actions, and vocal vibrations. The processed physiological and/or behavioral characteristics can then be used in connection with virtual conferences for various purposes, including for example, telehealth, physical fitness training, and gaming. For example, healthcare providers can be aware of asynchronous measurement of vitals or synchronous measurement of vitals during which they can view and interpret these characteristics in or near real-time.

The virtual conferencing system of the present disclosure is configured to extract a combination of physiological vital signs and/or behavioral cues from audio and/or visual media data and process the media data via both a computing device of the user and a remote device, such as a cloud server, associated with the virtual conferencing system to increase the accuracy of the measurements and efficiency of the virtual conferencing system. Specifically, first processing of the unprocessed, raw media data is performed on the user's computing device to extract intermediate user data prior to transmitting the media data to the server, and second processing is subsequently performed on the server to analyze the media data with the intermediate user data to measure user characteristics. In some aspects, physiological and/or behavioral user characteristics may be used as a vector to train machine learning models for performing predictive analytics on user information, such as recommending stress reduction, seeing a healthcare provider for cardiovascular purposes, or adjusting game parameters.

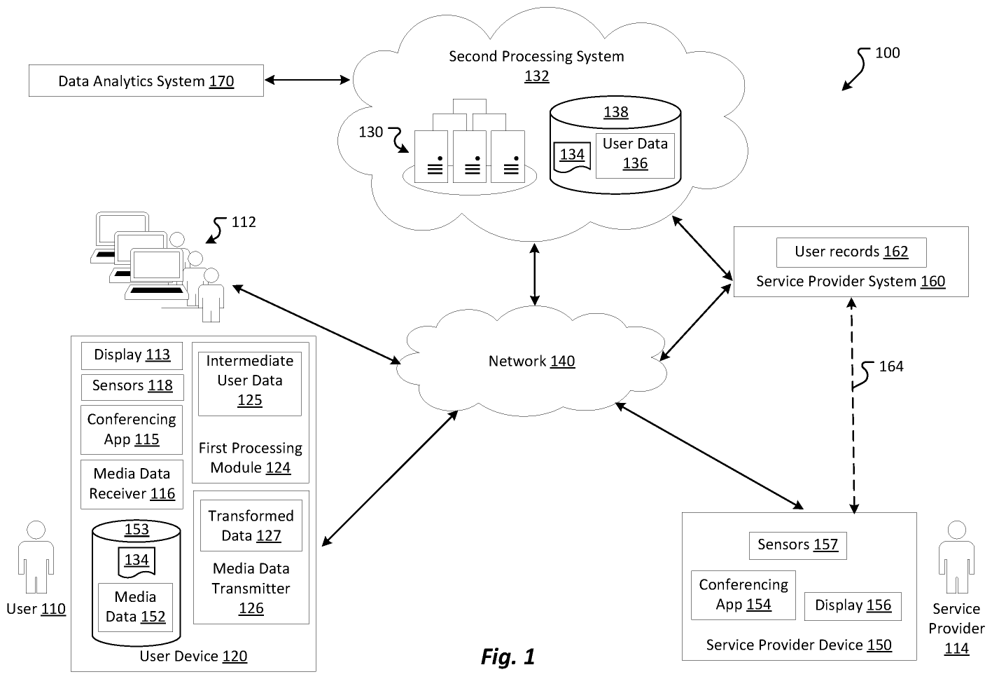

FIG. 1 depicts a virtual conferencing system 100 for remotely measuring physiological and/or behavioral characteristics associated with a user 110 in accordance with examples of the present disclosure. To do so, the virtual conferencing system 100 includes a computing device 120 associated with the user 110 and one or more remote devices, such as servers 130, that are communicatively coupled to the computing device 120 via a network 140. The network 140 may include any kind of computing network including, without limitation, a wired or wireless local area network (LAN), a wired or wireless wide area network (WAN), and/or the Internet.

The computing device 120 includes or is communicatively coupled to one or more sensors 118 that are capable of capturing or generating media data 152 (e.g., videos, audios, and/or images) of the user 110. The one or more sensors 118 include, but are not limited to, one or more cameras and/or one or more microphones. In aspects, the computing device 120 includes a media data receiver 116 that is configured to collect, generate, receive, or otherwise obtain media data 152 associated with the user 110 using the one or more sensors 118. Additionally, the computing device 120 includes a media data transmitter 126 that is configured to transmit the media data to another device (e.g., a server 130) for further analysis.

Based on the media data 152, the virtual conferencing system 100 is configured to remotely measure one or more physiological and/or behavioral characteristics of the user 110. For example, the physiological characteristics may include, without limitation, peripheral blood flow, heart rate, heart rate variability, respiration, blood oxygenation, blood pressure, facial actions, blushing, blinking, and/or body and vocal vibrations. The behavioral characteristics may include, without limitation, speaking pace, voice tones, eye movements, blinking, and/or body and vocal vibrations. As an example, the media data 152 may be a video and/or images that capture variations in color of the user's skin as the light is absorbed and reflected from the user's skin. The virtual conferencing system 100 may extract and detect the color variations of the user's skin to measure a heart rate and/or a blood oxygenation level of the user. An example of extraction and detection the color variations of skin is described in U.S. patent application Ser. No. 16/902,047 “Animating Physiological Characteristics on 2D or 3D Avatars,” filed Jun. 15, 2020, the entire disclosure of which is expressly incorporated by reference herein.

Additionally, or alternatively, the video may have captured motion of the user 110, which may be used to measure a breathing motion, respiration/breathing rate, vocal vibrations, body vibrations (e.g., uncontrollable motion, which may be related to a disease or symptom). In other examples, the media data 152 may be audio that tracks sounds associated with the user 110, which may be used to determine respiration information and/or vocalization features (e.g., variations in voice tones, voice features, and speaking pace).

The user computing device 120 further includes a conferencing app 115 and display 113 for conducting virtual conferences with other users, such as users 112 and/or service provider 114 over the network 140. Virtual conferences include, without limitation, teleconferences and video conferences. It should be understood that a virtual conference may be a conference held between two or more users. In some aspects, a virtual conference may be a conference held between one user and a virtual agent.

Service provider 114 includes, without limitation, any user (e.g., service provider 114) and/or system (e.g., service provider system 160) that provides a service to the user 110, such as a healthcare provider or a personal trainer, on the one hand, or a gaming system other hand. Service provider 114 may likewise have a service provider computing device 150 that includes sensors 157, a display 156, and a conferencing app 154 for conducting virtual conferences with users, such as user 110 and users 112, over the network 140. In aspects, the service provider computing device 150 may be directly connected via connection 164 to service provider system 160. Service provider system 160 may include user records 162, such as electronic medical records, physical fitness records, or gaming profiles. In aspects, the second processing system 132 may combine the user characteristics 136 with the user records 162 to create reports 134.

For example, during a virtual conference between a user 110 and a third-party service provider 114 (e.g., a healthcare provider and/or a fitness trainer), the media data 152 may be collected and analyzed during the virtual conference to provide the third-party service provider 114 with one or more physiological and/or behavioral characteristics of the user. This allows the third-party service provider 114 to view and interpret the physiological and/or behavioral characteristics of the user 110 during the virtual conference to provide services to the user 110. Additionally, or alternatively, the media data 152 may be collected and at least partially analyzed prior to the virtual conference with the third-party service provider 114. Additionally, the user 110 may identify one or more third-party service providers 114 who are authorized to access the user's data and may further indicate which physiological and/or behavioral characteristics of the user can be accessed by each of the third-party service providers 114.

Referring back to FIG. 1, once the media data 152 is collected by the sensors 118, the media data 152 may be stored in storage 153 on the user computing device 120 and/or may be collected by the media data receiver 116. The media data receiver 116 may collect the media data 152 directly from the sensors 118 and/or from the storage 153. A first processing module 124 may perform first processing to analyze the unprocessed, raw media data 152 to extract intermediate user data 125. The raw media data 152 is media data that has not been processed (e.g., not compressed). The intermediate user data 125 includes, without limitation, one or more physiological and/or behavioral signals associated with one or more physiological and/or behavioral characteristics of the user 110. As described further below, the intermediate user data 125 is transmitted to the second processing system 132 for further analysis.

In aspects, the physiological or behavioral signal may be a waveform corresponding to a frequency, duration, and/or amplitude associated with a physiological or behavioral characteristic of the user. For example, the physiological signal may correspond to a heart rate and may be provided as a heartrate waveform including systole, diastole, and dicrotic notch portions. Alternatively, or additionally, the physiological signal may correspond to a respiratory waveform, a blushing waveform, and/or a blinking waveform. Although specific waveforms have been identified, it should be understood that any physiological or behavioral characteristic that may be triggered in some fashion and/or duration may be provided as the physiological or behavioral signal that may be extracted from the media data. Similarly, the behavioral signal is indicative of one or more behavioral characteristics.

Subsequently, a media data transmitter 126 is configured to transform the raw media data 152 into a form, i.e., transformed media data 127, and transmit the transformed media data 127 with the intermediate user data 125 to the second processing system 132 for further analysis. For example, the media data transmitter 126 may compress the media data 152 and parse the compressed media data into smaller data packets to be transmitted to the second processing system 132. The transformation allows the media data to be efficiently transmitted to the second processing system 132 over the network 140 by increasing transfer speed and reducing resources that are required to store and transmit the media data. It should be appreciated that, in some aspects, the intermediate user data 125 may also be transformed into smaller data packets based on the size of the intermediate user data 125.

The second processing system 132 includes one or more servers 130 and one or more databases 138. The server(s) 130 may be cloud servers. Once the server 130 receives the transformed media data 127 and the intermediate user data 125, a second processing may be performed by the server 130 to further analyze the transformed media data 127 and the intermediate user data 125 to determine one or more physiological and/or behavioral characteristics 136 of the user 110. For example, the server 130 may derive one or more physiological and/or behavioral characteristics 136 based at least in part on the one or more physiological and/or behavioral signals included in the intermediate user data 125. Alternatively, or additionally, the server 130 may extract one or more physiological and/or behavioral characteristics 136 from the transformed media data 127. The server 130 may store the user characteristics 136 in the database 138. The server 130 may also create one or more reports 134 for user 110 and/or service provider 114 based on the user characteristics 136 and/or the intermediate user data 125. The reports 134 may be stored in database 138. Further, in some aspects, the report 134 may be sent back to the user 110 to be stored in database 153 on the user computing device 120.

Some information of the raw media data 152 may potentially be lost during the transformation and transmission from the user computing device 120 to the second processing system 132. As such, performing an initial analysis (e.g., first processing) by the first processing module 124 allows the virtual conferencing system 100 to accurately extract intermediate information 125 (e.g., physiological and/or behavioral signals) from the unprocessed, raw media data 152 prior to any potential data loss.

In one example, the computing device 120 may collect video data (i.e., media data 152) using a camera (i.e., sensor 118) that is communicatively coupled to the computing device 120. Once the video data 152 is collected, the first processing module 124 may extract a blood volume pulse signal (e.g., a pulse waveform), i.e., the intermediate user data 125, from the raw video frames of the video data 152. Additionally, the media data transmitter 126 may transform the video data 152 and transmit the transformed video data 127 with the intermediate user data 125 to the server 130. Subsequently, the server 130 may perform a second processing of the transformed media data 127 to derive a heart rate (i.e., user characteristic 136) of the user 110 from the blood volume pulse signal.

In another example, which may be in addition to the first example, the first processing module 124 may process the raw video data (i.e., media data 152) to extract a respiration signal (e.g., a respiration waveform), i.e., the intermediate user data 125, prior to the transformation and transmission of the video data to the server 130. In such an example, the server 130 may further perform a second processing of the transformed media data 127 to derive a respiration or breathing rate of the user from the respiration signal.

In aspects, the physiological and/or behavioral signals may be collected and measured synchronously (e.g., in or near real-time) during a virtual conference with a third-party service provider 114. For example, during a virtual appointment with a healthcare provider 114, media data 152 of the user 110 may be collected and used to measure one or more physiological and/or behavioral characteristics 136 of the user 110. This allows the healthcare provider 114 to examine the user 110 and interpret the physiological and/or behavioral characteristics 136 for diagnosis in or near real-time. However, it should be appreciated that, in some aspects, the physiological and/or behavioral characteristics 136 may be measured asynchronously with the virtual conference. For example, the media data 152 of the user 110 may be collected and analyzed before the virtual appointment with the healthcare provider 114. The user 110 may collect the media data 152 periodically prior to the virtual appointment, such that the healthcare provider 114 may view the progress of the user's physiological and/or behavioral characteristics 136 over a period of time and not in connection with a specific virtual conference.

In some aspects, the media data 152 of the user may be collected during a virtual session with a personal or smart trainer (i.e., service provider 114). In such an example, the personal or smart trainer may measure one or more physiological and/or behavioral characteristics 136 of the user throughout the virtual session in or near real-time to determine a fitness level of the user to suggest appropriate exercises.

In other aspects, the computing device 120 may be embodied as a game controller and the service provider 114 may be a gaming system (i.e., service provider system 160). In such aspects, the one or more physiological and/or behavioral characteristics 136 (e.g., the heart rate) of the user 110 may be measured when the user 110 is playing a game via the game controller 120 and subsequently used by the gaming system to suggest game parameters suitable for the user. For example, the game controller 120 may extract a heartrate waveform from the media data 152 collected from one or more sensors 118 of the game controller 120 and transmit the heartrate waveform to the server 130. The server 130 may process the heartrate waveform to measure the heart rate of the user. The server 130 may further transmit the heart rate to the gaming system (e.g., system 160). The gaming system may determine, for example, whether to adjust a difficulty level of the game based at least in part on the heart rate of the user.

In some aspects, the virtual conferencing system 100 may perform large-scale data analytics using data analytics system 170 to put physiological and/or behavioral characteristics 136 into context. For example, physiological and/or behavioral data of the user 110 and other users 112, either alone or in combination with user records 162 and/or user reports 134, may be used as a vector to train machine learning models for performing predictive analytics on user characteristics 136 as well as other information about users, such as recommending stress reduction, seeing a healthcare provider for cardiovascular purposes, or adjusting game parameters. In aspects, this information may be used to train other third-party service providers, such as service provider 114 and service provider system 160.

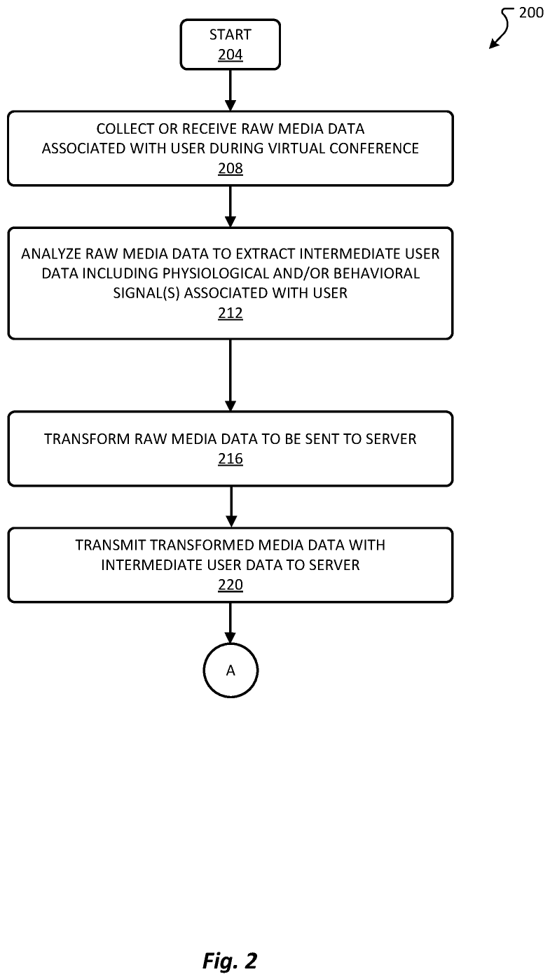

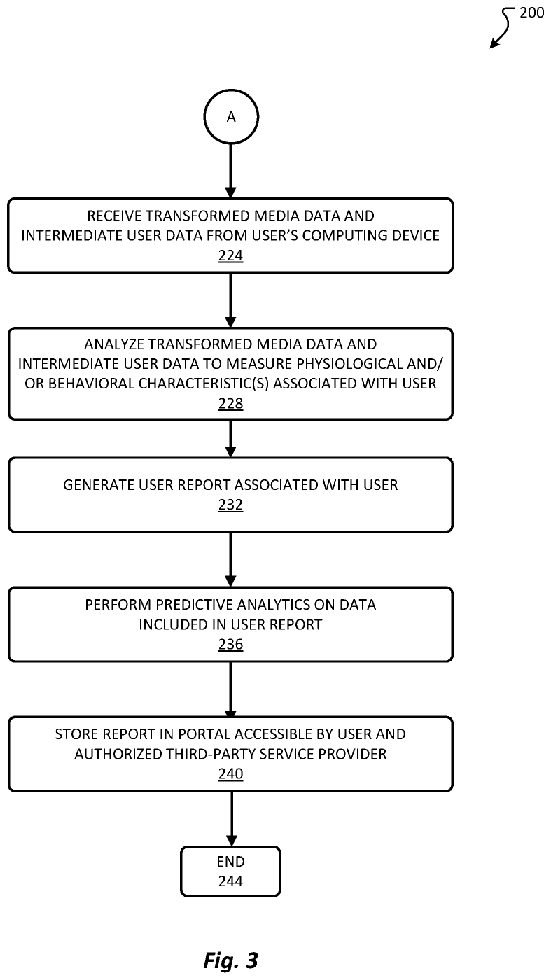

Referring now to FIGS. 2 and 3, a method 200 for providing user characteristics to a service provider for a virtual conference in accordance with examples of the present disclosure is provided. A general order for the steps of the method 200 is shown in FIGS. 2 and 3. Generally, the method 200 starts at 204 and ends at 244. The method 200 may include more or fewer steps or may arrange the order of the steps differently than those shown in FIGS. 2 and 3. In the illustrative aspect, the method 200 is performed in part by a computing device (e.g., a user device 120) of a user 110 (e.g., 208-220) and in part by a server (e.g., a server 130) associated with a virtual conferencing system (e.g., 224-240). For example, the computing device 120 may be, but not limited to, a computer, a notebook, a laptop, a mobile device, a smartphone, a tablet, wearable, or any other suitable computing device that is capable of communicating with the server 130. For example, the server 130 may be any suitable computing device that is capable of communicating with the computing device 120. The method 200 can be executed as a set of computer-executable instructions executed by a computer system and encoded or stored on a computer readable medium. Further, the method 200 can be performed by gates or circuits associated with a processor, Application Specific Integrated Circuit (ASIC), a field programmable gate array (FPGA), a system on chip (SOC), or other hardware device. Hereinafter, the method 200 shall be explained with reference to the systems, components, modules, software, data structures, user interfaces, etc. described in conjunction with FIG. 1.

The method 200 starts at 204, where flow may proceed to 208. It should be appreciated that 208-220 of the method 200 illustrate steps for performing a first processing of media data by a computing device (e.g., user device 120) of a user 110 to extract one or more physiological and/or behavioral signals associated with the user in accordance with examples of the present disclosure.

To do so, at operation 208, during a virtual conference between a user 110 and a third-party service provider 114 (e.g., a healthcare provider and/or a fitness trainer), the computing device 120 of the user collects, generates, receives, or otherwise obtains raw media data 152 associated with the user 110. Specifically, in the illustrative aspect, the computing device 120 collects media data 152 in or near real-time during the virtual conference using one or more sensors 118 (e.g., a camera and/or a microphone) that are included with or communicatively coupled to the computing device 120. For example, the media data 152 may include one or more videos, audios, and/or images of the user, and the raw media data represents original and unprocessed data that was received from the one or more sensors. As an example, the computing device 120 may collect video data using a camera that is communicatively coupled to the computing device 120. Additionally or alternatively, audio data may be collected from a microphone that is communicatively coupled to the computing device 120.

Once the raw media data is collected, the method 200 proceeds to operation 212. At operation 212, the computing device 120 analyzes the raw media data 152 to extract intermediate user data 125 (e.g., one or more physiological and/or behavioral signals associated with one or more physiological and/or behavioral characteristics of the user). As described above, the media data 152 may be analyzed during the virtual conference to allow the third-party service provider 114 to view and interpret one or more physiological and/or behavioral characteristics of the user in or near real-time.

For example, the physiological characteristics may include peripheral blood flow, heart rate, heart rate variability, respiration, blood oxygenation, blood pressure, facial actions, blushing, blinking, and/or body and vocal vibrations. The behavioral characteristics may include speaking pace, voice tones, eye movements, blinking, and/or body and vocal vibrations. As described above, a physiological and/or behavioral signal may be a waveform corresponding to a frequency, duration, and/or amplitude associated with a physiological and/or behavioral characteristic of the user. For example, the physiological signal may correspond to a heart rate and may be provided as a heartrate waveform including systole, diastole, and dicrotic notch portions. Alternatively, or additionally, the physiological signal may correspond to a respiratory waveform, a blushing waveform, and/or a blinking waveform. Although specific waveforms have been identified, it should be understood that any physiological characteristic that maybe triggered in some fashion and/or duration may be provided as the physiological signal that may be extracted from the media data.

The method 200 may then proceed to operation 216, where the raw media data 152 is transformed into a form, i.e., transformed media data 127, that allows it to be efficiently transmitted to a server (e.g., the server 130) over a network. To do so, for example, the computing device 120 may compress the raw media data 152 and parse the compressed raw media data 152 into smaller packets to be transmitted to the server 130.

At operation 220, the transformed media data 127 is transmitted to the server 130 with the intermediate user data 125 for further analysis. As an example, the computing device 120 may transmit the transformed media data 127 and the intermediate user data 125 to the server 130 via a virtual conference portal that is accessible by the user and the third-party service provider 114 via the conferencing app 115. As described above, the intermediate user data 125 includes the initial analysis of the raw media data 152 performed at the computing device 120 via the first processing by the computing device 120. Specifically, the intermediate user data includes the one or more physiological and/or behavioral signals associated with one or more physiological and/or behavioral characteristics of the user that are extracted from the raw media data 152. Because some information of the media data may potentially be lost during the transformation and transmission of the media data at operations 216 and 220, performing an initial analysis by the computing device 120 allows the virtual conferencing system 100 to accurately extract user information (e.g., physiological and/or behavioral signals) from the unprocessed, raw media data prior to any potential data loss.

Subsequently, the method 200 proceeds to operation 224 in FIG. 3 as shown by the alphanumeric character A in FIGS. 2 and 3. It should be appreciated that operations 224-240 of the method 200 illustrate steps for performing a second processing of media data by the server 130. To do so, at operation 224, the server 130 receives transformed media data 127 and the intermediate user data 125 from the computing device 120.

At operation 228, the server 130 performs the second processing of the transformed media data 127 and the intermediate user data 125 to further analyze the media data to measure one or more physiological and/or behavioral characteristics associated with the user. In the illustrative aspect, the server 130 may derive one or more physiological and/or behavioral characteristics from the one or more physiological and/or behavioral signals included in the intermediate user data 125. Alternatively, or additionally, the server 130 may extract one or more physiological and/or behavioral characteristics 136 from the transformed media data 127.

Additionally, or alternatively, the video and/or images may capture variations in color of the user's skin as the light is absorbed and reflected from the user's skin. The server 130 may measure the color variations of the user's skin to measure a heart rate and/or a blood oxygenation level of the user. Additionally, or alternatively, the video data may capture motion of the user, which may be used to measure a breathing motion, respiration/breathing rate, vocal vibrations, body vibrations (e.g., uncontrollable motion, which may be related to a disease or symptom). Additionally, or alternatively, the audio data may capture sounds associated with the user, which may be used to determine respiration information and/or vocalization features (e.g., variations in voice tones, voice features, and speaking pace).

At operation 232, the server 130 generates a user report associated with the user based on the physiological and/or behavioral characteristics. In some aspects, the server 130 may combine the physiological and/or behavioral characteristics with other data associated with the user. The other data may include a health-related record (e.g., a medical record) or any other data or record associated with the user, which may be received or obtained from a third-party source (e.g., a service provider system 160). By combining the measured physiological and/or behavioral characteristics of the user with other data may provide further insights to the user and the third-party service provider 114.

At operation 236, in some aspects, the server 130 may further perform predictive analytics on information included in the user report to provide a recommended action, such as recommending stress reduction, seeing a physician for cardiovascular purposes, and/or adjusting game parameters. In such aspects, the server 130 may update the user report to include the one or more recommended actions from the predictive analytics.

At operation 240, the server 130 stores the user report in the virtual conference portal, which is accessible by the user and the third-party service provider 114 via the conferencing app 115. As described above, the user 110 may assign an authorization level to the third-party service provider 114 prior to or during the virtual conference indicating which of the physiological and/or behavioral characteristics are accessible by the third-party service provider 114. For example, if the lowest authorization level is assigned to the third-party service provider 114, the third-party service provider 114 may have access to general physiological vitals (e.g., heart rate, blood pressure, and respiration rate) of the user 110. The behavioral characteristics of the user 110 may only be accessible to third-party service provider 114 that the highest authorization level. Based on the authorization level, the third-party service provider 114 may view and edit the user report to include a summary of the virtual conference, a contextualized data (e.g., an interpretation of the physiological and/or behavioral characteristics), and/or a recommendation (e.g., taking a medication, eating recommended food, and/or following a recommended exercise). In some aspects, the user report may be incorporated in the user's medical record. Method 200 may end at 244.

It should be appreciated that, in some aspects, the media data may be collected and analyzed prior to the virtual conference with the third-party service provider 114. For example, this allows the user to periodically upload media data indicative of user's physiological and/or behavioral characteristics. Such media data may be used to determine a progress of the user's physiological and/or behavioral characteristics. In such aspects, the first and second processing may be performed prior to or during the virtual conference with the third-party service provider 114. Additionally, the user may identify one or more third-party service providers who are authorized to access the user's data and may further indicate which physiological and/or behavioral characteristics can be accessed by each of the authorized third-party service providers.

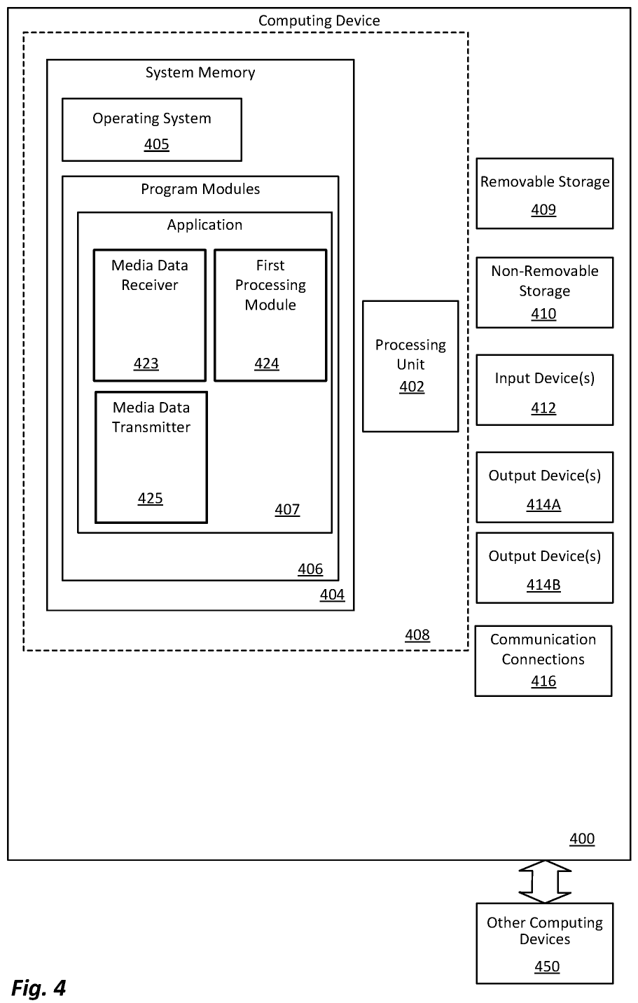

FIGS. 4-6 and the associated descriptions provide a discussion of a variety of operating environments in which aspects of the disclosure may be practiced. However, the devices and systems illustrated and discussed with respect to FIGS. 4-6 are for purposes of example and illustration and are not limiting of a vast number of computing device configurations that may be utilized for practicing aspects of the disclosure, described herein.

FIG. 4 is a block diagram illustrating physical components (e.g., hardware) of a computing device 400 with which aspects of the disclosure may be practiced. The computing device components described below may be suitable for the computing devices described above. For example, the computing device 400 may represent the computing device 120 of FIG. 1. In a basic configuration, the computing device 400 may include at least one processing unit 402 and a system memory 404. Depending on the configuration and type of computing device, the system memory 404 may comprise, but is not limited to, volatile storage (e.g., random access memory), non-volatile storage (e.g., read-only memory), flash memory, or any combination of such memories.

The system memory 404 may include an operating system 405 and one or more program modules 406 suitable for performing the various aspects disclosed herein such. The operating system 405, for example, may be suitable for controlling the operation of the computing device 400. Furthermore, aspects of the disclosure may be practiced in conjunction with a graphics library, other operating systems, or any other application program and is not limited to any particular application or system. This basic configuration is illustrated in FIG. 4 by those components within a dashed line 408. The computing device 400 may have additional features or functionality. For example, the computing device 400 may also include additional data storage devices (removable and/or non-removable) such as, for example, magnetic disks, optical disks, or tape. Such additional storage is illustrated in FIG. 4 by a removable storage device 409 and a non-removable storage device 410.

As stated above, several program modules and data files may be stored in the system memory 404. While executing on the at least one processing unit 402, the program modules 406 may perform processes including, but not limited to, one or more aspects, as described herein. The application 420 includes a media data receiver 423, a first processing module 424, and a media data transmitter 425, as described in more detail with regard to FIG. 1. Other program modules that may be used in accordance with aspects of the present disclosure may include electronic mail and contacts applications, word processing applications, spreadsheet applications, database applications, slide presentation applications, drawing or computer-aided application programs, etc., and/or one or more components supported by the systems described herein.

Furthermore, aspects of the disclosure may be practiced in an electrical circuit comprising discrete electronic elements, packaged or integrated electronic chips containing logic gates, a circuit utilizing a microprocessor, or on a single chip containing electronic elements or microprocessors. For example, aspects of the disclosure may be practiced via a system-on-a-chip (SOC) where each or many of the components illustrated in FIG. 4 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the computing device 400 on the single integrated circuit (chip). Aspects of the disclosure may also be practiced using other technologies capable of performing logical operations such as, for example, AND, OR, and NOT, including but not limited to mechanical, optical, fluidic, and quantum technologies. In addition, aspects of the disclosure may be practiced within a general-purpose computer or in any other circuits or systems.

The computing device 400 may also have one or more input device(s) 412 such as a keyboard, a mouse, a pen, a sound or voice input device, a touch or swipe input device, etc. The output device(s) 414A such as a display, speakers, a printer, etc. may also be included. An output 414B, corresponding to a virtual display may also be included. The aforementioned devices are examples and others may be used. The computing device 400 may include one or more communication connections 416 allowing communications with other computing devices 450. Examples of suitable communication connections 416 include, but are not limited to, radio frequency (RF) transmitter, receiver, and/or transceiver circuitry; universal serial bus (USB), parallel, and/or serial ports.

The term computer readable media as used herein may include computer storage media. Computer storage media may include volatile and nonvolatile, removable and non-removable media implemented in any method or technology for storage of information, such as computer readable instructions, data structures, or program modules. The system memory 404, the removable storage device 409, and the non-removable storage device 410 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 400. Any such computer storage media may be part of the computing device 400. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

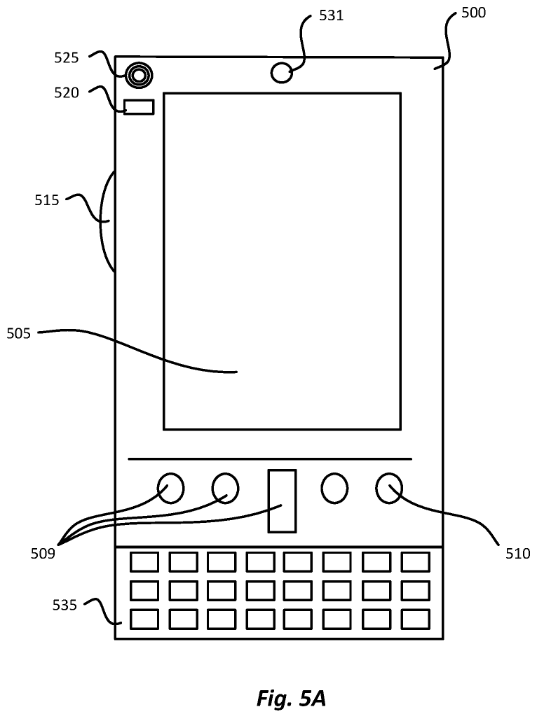

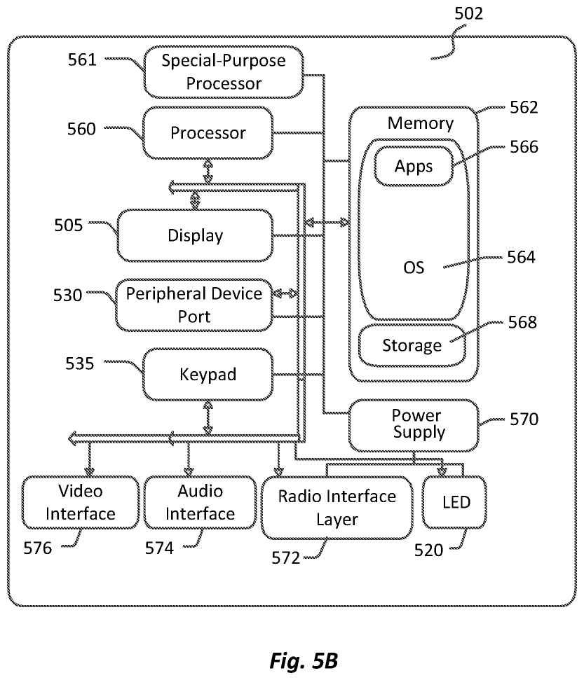

FIGS. 5A and 5B illustrate a computing device or mobile computing device 500, for example, a mobile telephone, a smart phone, wearable computer (such as a smart watch), a tablet computer, a laptop computer, and the like, with which aspects of the disclosure may be practiced. With reference to FIG. 5A, one aspect of a mobile computing device 500 for implementing the aspects is illustrated. In a basic configuration, the mobile computing device 500 is a handheld computer having both input elements and output elements. The mobile computing device 500 typically includes a display 505 and one or more input buttons 509/510 that allow the user to enter information into the mobile computing device 500. The display 505 of the mobile computing device 500 may also function as an input device (e.g., a touch screen display). If included, an optional side input element 515 allows further user input. The side input element 515 may be a rotary switch, a button, or any other type of manual input element. In alternative aspects, mobile computing device 500 may incorporate more or less input elements. For example, the display 505 may not be a touch screen in some aspects. In yet another alternative aspect, the mobile computing device 500 is a portable phone system, such as a cellular phone. The mobile computing device 500 may also include an optional keypad 535. Optional keypad 535 may be a physical keypad or a “soft” keypad generated on the touch screen display. In various aspects, the output elements include the display 505 for showing a graphical user interface (GUI), a visual indicator 531 (e.g., a light emitting diode), and/or an audio transducer 525 (e.g., a speaker). In some aspects, the mobile computing device 500 incorporates a vibration transducer for providing the user with tactile feedback. In yet another aspect, the mobile computing device 500 incorporates input and/or output ports 530, such as an audio input (e.g., a microphone jack), an audio output (e.g., a headphone jack), and a video output (e.g., a HDMI port) for sending signals to or receiving signals from an external source.

FIG. 5B is a block diagram illustrating the architecture of one aspect of computing device, a server, or a mobile computing device. That is, the mobile computing device 500 can incorporate a system (902) (e.g., an architecture) to implement some aspects. The system 502 can implemented as a “smart phone” capable of running one or more applications (e.g., browser, e-mail, calendaring, contact managers, messaging clients, games, and media clients/players). In some aspects, the system 502 is integrated as a computing device, such as an integrated personal digital assistant (PDA) and wireless phone.

One or more application programs 566 may be loaded into the memory 562 and run on or in association with the operating system 564. Examples of the application programs include phone dialer programs, e-mail programs, personal information management (PIM) programs, word processing programs, spreadsheet programs, Internet browser programs, messaging programs, and/or one or more components supported by the systems described herein. The system 502 also includes a non-volatile storage area 568 within the memory 562. The non-volatile storage area 568 may be used to store persistent information that should not be lost if the system 502 is powered down. The application programs 566 may use and store information in the non-volatile storage area 568, such as e-mail or other messages used by an e-mail application, and the like. A synchronization application (not shown) also resides on the system 502 and is programmed to interact with a corresponding synchronization application resident on a host computer to keep the information stored in the non-volatile storage area 568 synchronized with corresponding information stored at the host computer. As should be appreciated, other applications may be loaded into the memory 562 and run on the mobile computing device 500 described herein (e.g. a media data receiver 423, a first processing module 424, a media data transmitter 425, etc.).

The system 502 has a power supply 570, which may be implemented as one or more batteries. The power supply 570 might further include an external power source, such as an AC adapter or a powered docking cradle that supplements or recharges the batteries.

The system 502 may also include a radio interface layer 572 that performs the function of transmitting and receiving radio frequency communications. The radio interface layer 572 facilitates wireless connectivity between the system 502 and the “outside world,” via a communications carrier or service provider. Transmissions to and from the radio interface layer 572 are conducted under control of the operating system 564. In other words, communications received by the radio interface layer 572 may be disseminated to the application programs 566 via the operating system 564, and vice versa.

The visual indicator 520 may be used to provide visual notifications, and/or an audio interface 574 may be used for producing audible notifications via the audio transducer 525. In the illustrated configuration, the visual indicator 520 is a light emitting diode (LED) and the audio transducer 525 is a speaker. These devices may be directly coupled to the power supply 570 so that when activated, they remain on for a duration dictated by the notification mechanism even though the processor 560/961 and other components might shut down for conserving battery power. The LED may be programmed to remain on indefinitely until the user takes action to indicate the powered-on status of the device. The audio interface 574 is used to provide audible signals to and receive audible signals from the user. For example, in addition to being coupled to the audio transducer 525, the audio interface 574 may also be coupled to a microphone to receive audible input, such as to facilitate a telephone conversation. In accordance with aspects of the present disclosure, the microphone may also serve as an audio sensor to facilitate control of notifications, as will be described below. The system 502 may further include a video interface 576 that enables an operation of an on-board camera to record still images, video stream, and the like.

A mobile computing device 500 implementing the system 502 may have additional features or functionality. For example, the mobile computing device 500 may also include additional data storage devices (removable and/or non-removable) such as, magnetic disks, optical disks, or tape. Such additional storage is illustrated in FIG. 5B by the non-volatile storage area 568.

Data/information generated or captured by the mobile computing device 500 and stored via the system 502 may be stored locally on the mobile computing device 500, as described above, or the data may be stored on any number of storage media that may be accessed by the device via the radio interface layer 572 or via a wired connection between the mobile computing device 500 and a separate computing device associated with the mobile computing device 500, for example, a server computer in a distributed computing network, such as the Internet. As should be appreciated such data/information may be accessed via the mobile computing device 500 via the radio interface layer 572 or via a distributed computing network. Similarly, such data/information may be readily transferred between computing devices for storage and use according to well-known data/information transfer and storage means, including electronic mail and collaborative data/information sharing systems.

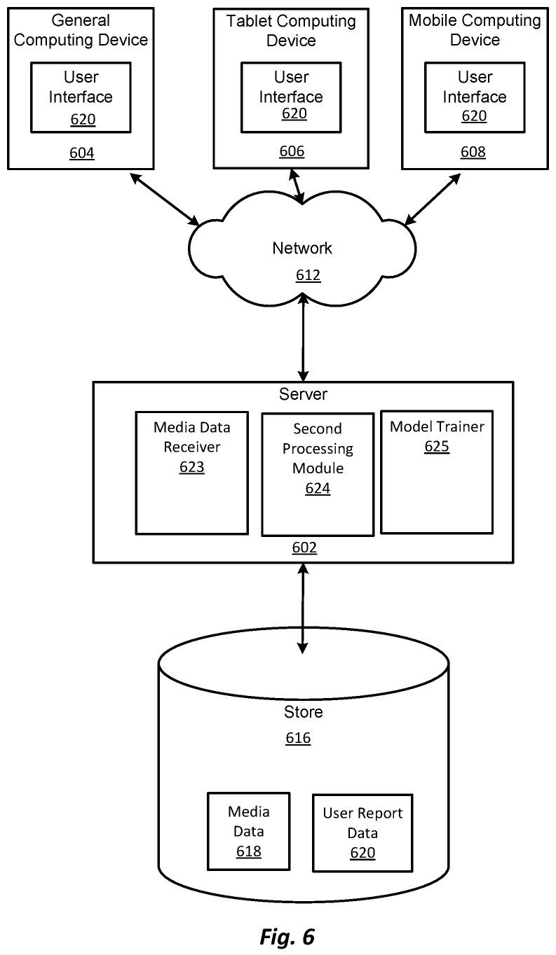

FIG. 6 illustrates one aspect of the architecture of a system for processing data received at a computing system from a remote source, such as a personal computer 604, tablet computing device 606, or mobile computing device 608, as described above. Content displayed at server device 602 may be stored in different communication channels or other storage types. For example, the computing device 604, 606, 608 may represent the computing device 120 of FIG. 1, and the server device 602 may represent the server 130 of FIG. 1.

In some aspects, one or more of a media data receiver 623, a second processing module 624, and a model trainer 625, may be employed by server device 602. The server device 602 may provide data to and from a client computing device such as a personal computer 604, a tablet computing device 606 and/or a mobile computing device 608 (e.g., a smart phone) through a network 612. By way of example, the computer system described above may be embodied in a personal computer 604, a tablet computing device 606 and/or a mobile computing device 608 (e.g., a smart phone). Any of these aspects of the computing devices may obtain content from the store 616, in addition to receiving graphical data useable to be either pre-processed at a graphic-originating system, or post-processed at a receiving computing system. The content store may include media data 618 and user report data 620.

FIG. 6 illustrates an exemplary mobile computing device 608 that may execute one or more aspects disclosed herein. In addition, the aspects and functionalities described herein may operate over distributed systems (e.g., cloud-based computing systems), where application functionality, memory, data storage and retrieval and various processing functions may be operated remotely from each other over a distributed computing network, such as the Internet or an intranet. User interfaces and information of various types may be displayed via on-board computing device displays or via remote display units associated with one or more computing devices. For example, user interfaces and information of various types may be displayed and interacted with on a wall surface onto which user interfaces and information of various types are projected. Interaction with the multitude of computing systems with which aspects of the invention may be practiced include, keystroke entry, touch screen entry, voice or other audio entry, gesture entry where an associated computing device is equipped with detection (e.g., camera) functionality for capturing and interpreting user gestures for controlling the functionality of the computing device, and the like.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Any of the steps, functions, and operations discussed herein can be performed continuously and automatically.

The exemplary systems and methods of this disclosure have been described in relation to computing devices. However, to avoid unnecessarily obscuring the present disclosure, the preceding description omits several known structures and devices. This omission is not to be construed as a limitation. Specific details are set forth to provide an understanding of the present disclosure. It should, however, be appreciated that the present disclosure may be practiced in a variety of ways beyond the specific detail set forth herein.

Furthermore, while the exemplary aspects illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed configurations and aspects.

Several variations and modifications of the disclosure can be used. It would be possible to provide for some features of the disclosure without providing others.

In yet another configurations, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another configuration, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another configuration, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

The disclosure is not limited to standards and protocols if described. Other similar standards and protocols not mentioned herein are in existence and are included in the present disclosure. Moreover, the standards and protocols mentioned herein, and other similar standards and protocols not mentioned herein are periodically superseded by faster or more effective equivalents having essentially the same functions. Such replacement standards and protocols having the same functions are considered equivalents included in the present disclosure.

In accordance with at least one example of the present disclosure, a method for providing a user characteristic to a service provider for a virtual conference with a user is provided. The method may include collecting, by a user computing device, raw media data associated with the user during the virtual conference between the user and the service provider, performing, by the user computing device during the virtual conference, a first processing of the raw media data to extract intermediate user data, wherein the intermediate user data comprises one or more of a physiological signal and a behavioral signal associated the user, transforming, by the computing device, the raw media data into transformed media data, and transmitting, by the computing device, the transformed media data with the intermediate user data to a server for second processing of the intermediate user data.

In accordance with at least one aspect of the above method, the method may include where the one or more of a physiological signal and a behavioral signal is a waveform corresponding to one or more of a frequency, a duration, and an amplitude associated with the user characteristic. In accordance with at least one aspect of the above method, the method may include where collecting the raw media data associated with the user further comprises collecting, by the computing device, raw media data using a sensor communicatively coupled to the user computing device. In accordance with at least one aspect of the above method, the method may include performing, by the server during the virtual conference, the second processing of the transformed media data to extract one or more of a physiological characteristic and a behavioral characteristic based at least in part upon the intermediate user data. In accordance with at least one aspect of the above method, the method may include where the one or more of a physiological characteristic and a behavioral characteristic comprises a plurality of user characteristics. In accordance with at least one aspect of the above method, the method may include assigning, by the computing device, an authorization level to the service provider indicating which of the plurality of user characteristics are accessible by the service provider. In accordance with at least one aspect of the above method, the method may include where the service provider includes at least one of a healthcare provider or a fitness trainer. In accordance with at least one aspect of the above method, the method may include where the computing device is a game controller and where collecting the raw media data associated with the user during the virtual conference between the user and the service provider further comprises collecting raw media data associated with the user during a game played by the user on a game server. In accordance with at least one aspect of the above method, the method may include where the raw media data comprises one or more of a video, an image, and audio, and wherein the one or more of a physiological signal and a behavioral signal comprise one or more of peripheral blood flow, heart rate, heart rate variability, respiration, blood oxygenation, blood pressure, facial actions, blushing, blinking, and body and vocal vibrations.

In accordance with at least one example of the present disclosure, a method for providing a user characteristic to a service provider for a virtual conference with a user is provided. The method may include collecting, by a user computing device, media data associated with the user, performing, by the user computing device, a first processing of the media data to extract intermediate user data, transmitting, by the user computing device, the media data with the intermediate user data to a server, performing, by the server, a second processing of the media data and the intermediate user data to generate the user characteristic, and providing, by the server, the user characteristic to the service provider for use in the virtual conference.

In accordance with at least one aspect of the above method, the method may include where transmitting the media data further comprises transforming, by the computing device, the media data into transformed media data, and transmitting, by the computing device, the transformed media data with the intermediate user data to the server, wherein performing the second processing of the media data further comprises performing, by the server, a second processing of the transformed media data to generate the user characteristic based at least in part on the intermediate user data. In accordance with at least one aspect of the above method, the method may include where the intermediate user data comprises one or more of a physiological signal and a behavioral signal associated with the user. In accordance with at least one aspect of the above method, the method may include where one or more of a physiological signal and a behavioral signal associated with the user comprises a waveform corresponding to the user characteristic. In accordance with at least one aspect of the above method, the method may include generating, by the server, a report based at least in part on the extracted one or more of a physiological characteristic and a behavioral characteristic, the report being accessible by the user and the service provider. In accordance with at least one aspect of the above method, the method may include where collecting the media data associated with the user comprises collecting media data asynchronously with the virtual conference. In accordance with at least one aspect of the above method, the method may include where the computing device is a game controller, and where collecting the media data associated with the user during the virtual conference between the user and the service provider further comprises collecting media data associated with the user during a game played by the user on a game server. In accordance with at least one aspect of the above method, the method may include where the service provider comprises at least one of a healthcare provider and a fitness trainer, and the media data comprises a video, an image, and audio, and wherein the one or more of a physiological signal and a behavioral signal comprises peripheral blood flow, heart rate, heart rate variability, respiration, blood oxygenation, blood pressure, facial actions, blushing, blinking, and/or body and vocal vibrations.

In accordance with at least one example of the present disclosure, a system for providing a user characteristic to a service provider for a virtual conference with a user is provided. The system may include a computing device and a server. The computing device may be configured to collect media data associated with the user, perform a first processing of the media data to extract intermediate user data, the intermediate user data comprising one or more of a physiological signal and a behavioral signal associated with the user, transmit the media data with the intermediate user data to a server. The server may be configured to perform a second processing of the media data and the intermediate user data to generate the user characteristic and provide the user characteristic to the service provider for use in the virtual conference.

In accordance with at least one aspect of the above system, the computing device may be further configured to transform the media data into transformed media data and transmit the transformed media data with the intermediate user data to the server, where the server is further configured to perform the second processing of the transformed media data to generate the user characteristic based at least in part on the intermediate user data. In accordance with at least one aspect of the above system, the server may be further configured to generate a report based at least in part on the extracted one or more of a physiological characteristic and a behavioral characteristic, the report being accessible by the user and the service provider. In accordance with at least one aspect of the above system, the computing device may be further configured to assign an authorization level to the service provider indicating which one or more of a physiological characteristic and a behavioral characteristic are accessible by the service provider.

The present disclosure, in various configurations and aspects, includes components, methods, processes, systems and/or apparatus substantially as depicted and described herein, including various combinations, subcombinations, and subsets thereof. Those of skill in the art will understand how to make and use the systems and methods disclosed herein after understanding the present disclosure. The present disclosure, in various configurations and aspects, includes providing devices and processes in the absence of items not depicted and/or described herein or in various configurations or aspects hereof, including in the absence of such items as may have been used in previous devices or processes, e.g., for improving performance, achieving ease, and/or reducing cost of implementation.

1. A method for providing a user characteristic to a service provider for a virtual conference with a user, the method comprising:
collecting, by a user computing device, raw media data associated with the user during the virtual conference between the user and the service provider;
performing, by the user computing device during the virtual conference, a first processing of the raw media data to extract intermediate user data, wherein the intermediate user data comprises one or more of a physiological signal and a behavioral signal associated the user;
transforming, by the computing device, the raw media data into transformed media data; and
transmitting, by the computing device, the transformed media data with the intermediate user data to a remote device for second processing of the intermediate user data.

2. The method of claim 1, wherein the one or more of a physiological signal and a behavioral signal is a waveform corresponding to one or more of a frequency, a duration, and an amplitude associated with the user characteristic.

3. The method of claim 1, wherein collecting the raw media data associated with the user further comprises collecting, by the computing device, raw media data using a sensor communicatively coupled to the user computing device.

4. The method of claim 1, further comprising:
performing, by the remote device during the virtual conference, the second processing of the transformed media data to extract one or more of a physiological characteristic and a behavioral characteristic based at least in part upon the intermediate user data.

5. The method of claim 1, wherein the one or more of a physiological characteristic and a behavioral characteristic comprises a plurality of user characteristics and the method further comprises:
assigning, by the computing device, an authorization level to the service provider indicating which of the plurality of user characteristics are accessible by the service provider.

6. The method of claim 1, wherein the service provider includes at least one of a healthcare provider or a fitness trainer.

7. The method of claim 1, wherein the computing device is a game controller, and wherein collecting the raw media data associated with the user during the virtual conference between the user and the service provider further comprises collecting raw media data associated with the user during a game played by the user on a game server.

8. The method of claim 1, wherein the raw media data comprises one or more of a video, an image, and audio, and wherein the one or more of a physiological signal and a behavioral signal comprise one or more of peripheral blood flow, heart rate, heart rate variability, respiration, blood oxygenation, blood pressure, facial actions, blushing, blinking, and body and vocal vibrations.

9. A method for providing a user characteristic to a service provider for a virtual conference with a user, the method comprising:
collecting, by a user computing device, media data associated with the user;
performing, by the user computing device, a first processing of the media data to extract intermediate user data;
transmitting, by the user computing device, the media data with the intermediate user data to a server;
performing, by the server, a second processing of the media data and the intermediate user data to generate the user characteristic; and
providing, by the server, the user characteristic to the service provider for use in the virtual conference.

10. The method of claim 9, wherein transmitting the media data further comprises:
transforming, by the computing device, the media data into transformed media data; and
transmitting, by the computing device, the transformed media data with the intermediate user data to the server, wherein performing the second processing of the media data further comprises performing, by the server, a second processing of the transformed media data to generate the user characteristic based at least in part on the intermediate user data.

11. The method of claim 9, wherein the intermediate user data comprises one or more of a physiological signal and a behavioral signal associated with the user.

12. The method of claim 11, wherein one or more of a physiological signal and a behavioral signal associated with the user comprises a waveform corresponding to the user characteristic.

13. The method of claim 9, further comprising generating, by the server, a report based at least in part on the extracted one or more of a physiological characteristic and a behavioral characteristic, the report being accessible by the user and the service provider.

14. The method of claim 9, wherein collecting the media data associated with the user comprises collecting media data asynchronously with the virtual conference.

15. The method of claim 9, wherein the computing device is a game controller, and wherein collecting the media data associated with the user during the virtual conference between the user and the service provider further comprises collecting media data associated with the user during a game played by the user on a game server.

16. The method of claim 11, wherein the service provider comprises at least one of a healthcare provider and a fitness trainer, and the media data comprises a video, an image, and audio, and wherein the one or more of a physiological signal and a behavioral signal comprises peripheral blood flow, heart rate, heart rate variability, respiration, blood oxygenation, blood pressure, facial actions, blushing, blinking, and/or body and vocal vibrations.

17. A system for providing a user characteristic to a service provider for a virtual conference with a user, the system comprising:
a computing device configured to:
collect media data associated with the user;
perform a first processing of the media data to extract intermediate user data, the intermediate user data comprising one or more of a physiological signal and a behavioral signal associated with the user;
transmit the media data with the intermediate user data to a server; and
the server configured to:
perform a second processing of the media data and the intermediate user data to generate the user characteristic; and
provide the user characteristic to the service provider for use in the virtual conference.

18. The system of claim 17, wherein computing device is further configured to:
transform the media data into transformed media data; and
transmit the transformed media data with the intermediate user data to the server, wherein the server is further configured to perform the second processing of the transformed media data to generate the user characteristic based at least in part on the intermediate user data.

19. The system of claim 17, wherein the server is further configured to:
generate a report based at least in part on the extracted one or more of a physiological characteristic and a behavioral characteristic, the report being accessible by the user and the service provider.

20. The system of claim 17, wherein the computing device is further configured to:
assign an authorization level to the service provider indicating which one or more of a physiological characteristic and a behavioral characteristic are accessible by the service provider.
